(19) United States
(12) Patent Application Publication
(10) Pub. No.: US 2019/0147727 A1
(43) Pub. Date: May 16, 2019

(54) System and Method for Monitoring Sanitation Solutions

(71) Applicants: James Koursaris, Naperville, IL (US); Paul Wieczorek, Livonia, MI (US)

(72) Inventors: James Koursaris, Naperville, IL (US); Paul Wieczorek, Livonia, MI (US)

(21) Appl. No.: 16/156,659

(22) Filed: Oct. 10, 2018

Related U.S. Application Data
(60) Provisional application No. 62/586,289, filed on Nov. 15, 2017.

(51) Int. Cl. G08B 21/18; H04Q 9/00; G08B 5/36; A61L 2/24

(52) U.S. Cl. CPC G08B 21/182; H04Q 9/00; A61L 2202/14; A61L 2/24; G08B 5/36

(57) ABSTRACT

A system and method for monitoring the concentration of sanitation solutions measures the concentration for a sanitation compound of a sanitation solution to ensure the presence of an effective quantity of the sanitation compound to reduce or eliminate infectious microorganisms from a desired surface. The system includes a concentration monitoring device to measure the concentration of the sanitation solution. This measurement is assessed against a pre-determined sanitizer concentration range using a microcontroller of the concentration monitoring device. If the measured concentration value deviates from the pre-determined sanitizer concentration range, the concentration monitoring device emits a visual or auditory alert to notify any near by persons to address the deviation in concentration. The concentration monitoring device is capable of notifying a remote computing device to alert relevant persons that are not able to immediately observe the concentration monitoring device of the deviation from the pre-determined sanitizer concentration range.

The current application claims a priority to the U.S. Provisional Patent application Ser. No. 62/586,289 filed on Nov. 15, 2017.

### FIELD OF THE INVENTION

The present invention relates generally to sanitation in the food processing industry. More specifically, the present invention relates to monitoring a sanitation solution to maintain an acceptable concentration for an active compound of the sanitation solution.

### BACKGROUND OF THE INVENTION

Sanitation is an important aspect of the food processing industry for reducing or eliminating infectious microorganisms. Infectious microorganisms pose a health safety risk. Food comes into contact with a plurality of surfaces, such as a kitchen table, during a plurality of food processes where infectious microorganisms may be present. As food comes into contact with such surfaces, some infections microorganisms migrate onto the food and, therefore, present as the food is consumed. The infectious microorganisms may cause the person that consumes the food containing the microorganisms to become sick or worse. Therefore, sanitation of the surfaces that come in contact with the food is important to reduce health hazards for persons consuming food prepared on these surfaces.

There is a plurality of sanitation solutions that may be used for sanitation to reduce infection organisms on a plurality of surfaces. Examples of sanitation solutions include quaternary ammonium compounds. However, the sanitation solutions lose their effectiveness as the sanitation solutions come in contact with air or are diluted with water. For example, some sanitation solutions lose their effectiveness in under 4 hours as the active compound evaporates or reacts with compounds in the air. However, the user of the sanitation solution is often unaware of the decline in effectiveness of the sanitation solutions. Therefore, the user may attempt to sanitize surfaces with ineffective sanitation solutions and have little impact on the presence of infectious microorganisms. Further, the user may not be able to comply with some compliances, such as sanitation compliances for restaurants, if the sanitation solution concentration becomes ineffective. For example, a sanitation solution may have a concentration range between 0 parts per million (PPM) and 500 PPM, although a governing health department may prescribe 200

PPM as the lower limit to ensure the sanitation solution is effective. So, if the concentration of the sanitation solution goes below 200 PPM, then the solution is deemed to be ineffective and will need to have additional sanitation compounds added to the solution.

The present invention is a system and method for monitoring the concentration of sanitation solutions. The present invention implements a concentration sensor in order to monitor the concentration for a sanitation solution to ensure the concentration of active compounds of the sanitation solution is within an effective range to reduce infectious microorganisms on surfaces the sanitation solution is applied. If the sanitation solution concentration deviates from a predetermined concentration range, the user is notified to add additional sanitation compounds to the sanitation solution to rectify the deviation.

### BRIEF DESCRIPTION OF THE DRAWINGS

FIG. 1 is a perspective view of the simplest embodiment for the system of the present invention.

FIG. 2 is a schematic diagram for the concentration monitoring device of the present invention, wherein a wireless transceiver is communicatively coupled with a remote computing device.

FIG. 3 is a flow diagram for the general process of the method of the present invention.

FIG. 4 is a more specific flow diagram for the method of the present invention, wherein the present invention comprises an auditory indicator.

FIG. 5 is a more specific flow diagram for the method of the present invention, wherein the present invention comprises a visual indicator.

FIG. 6 is a more specific flow diagram for the method of the present invention, wherein the present invention comprises a wireless transceiver.

### DETAIL DESCRIPTIONS OF THE INVENTION

All illustrations of the drawings are for the purpose of describing selected versions of the present invention and are not intended to limit the scope of the present invention.

The present invention is a system and method for monitoring a sanitation solution. The present invention allows a user to ensure a concentration of the sanitation solution is within acceptable bounds to reduce or eliminate infectious microorganisms. The present invention alerts the user if a sanitizer concentration value deviates from a pre-determined sanitizer concentration range. The pre-determined sanitizer concentration range is a range which a quantity of sanitation compounds within the sanitation solution is effective to reduce or eliminate the infectious microorganisms on a desired food surface to promote safe health practices.

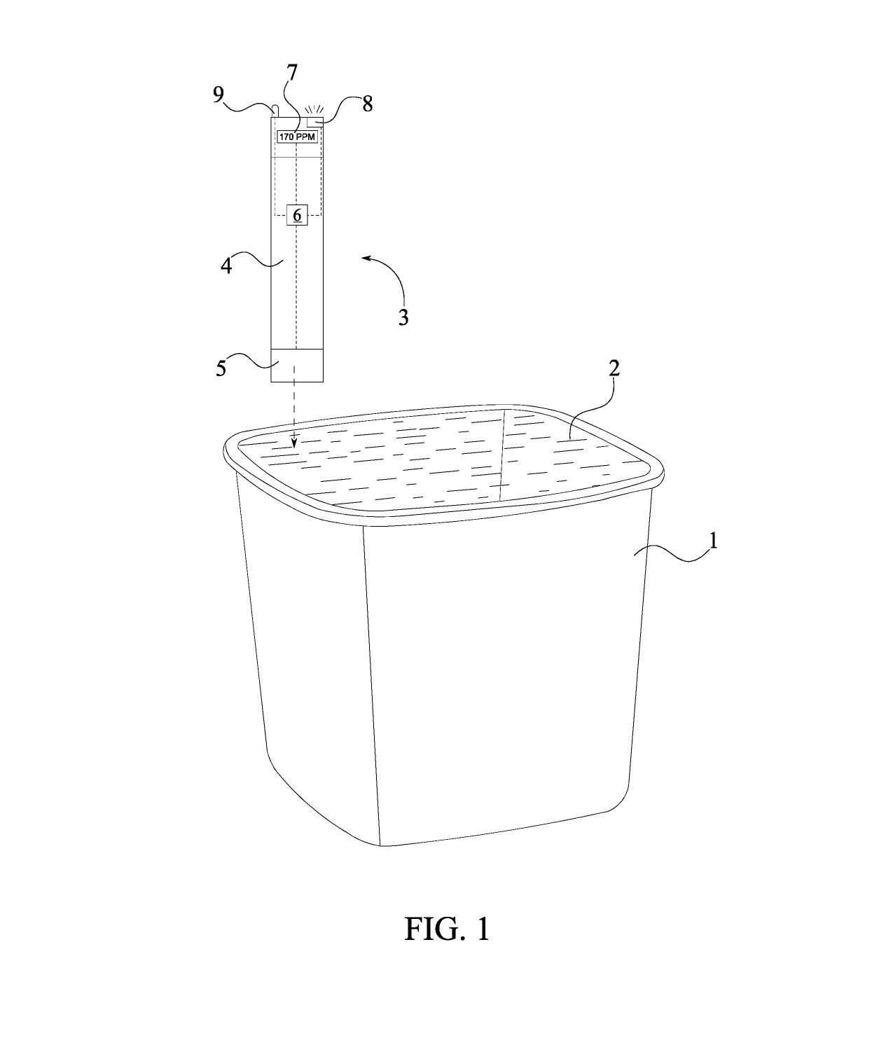

In accordance to the system for monitoring sanitation solutions, the present invention comprises a sanitation bucket 1, a sanitation solution 2, and a concentration monitoring device 3, detailed in FIG. 1. The sanitation bucket 1 is a container that the sanitation solution 2 is stored, such that the sanitation solution 2 is able to be accessed by a user to apply the sanitation solution 2 to the desired surface. The sanitation solution 2 is a disinfectant or surfactant utilized to remove infectious microorganisms from a plurality of surfaces. The sanitation solution 2 is preferred to be a quaternary ammonia compound solution; however, the sanitation solution 2 may be any appropriate surfactant, disinfectant, or sanitizer to clean a surface, to reduce infection microorganism on the surface, or to eliminate the present of infectious microorganisms from the surface. The sanitation bucket 1 is filled with the sanitation solution 2, such that the sanitation solution 2 contained within the sanitation bucket 1. The concentration monitoring device 3 measures the concentration of the active compounds within the sanitation solution 2.

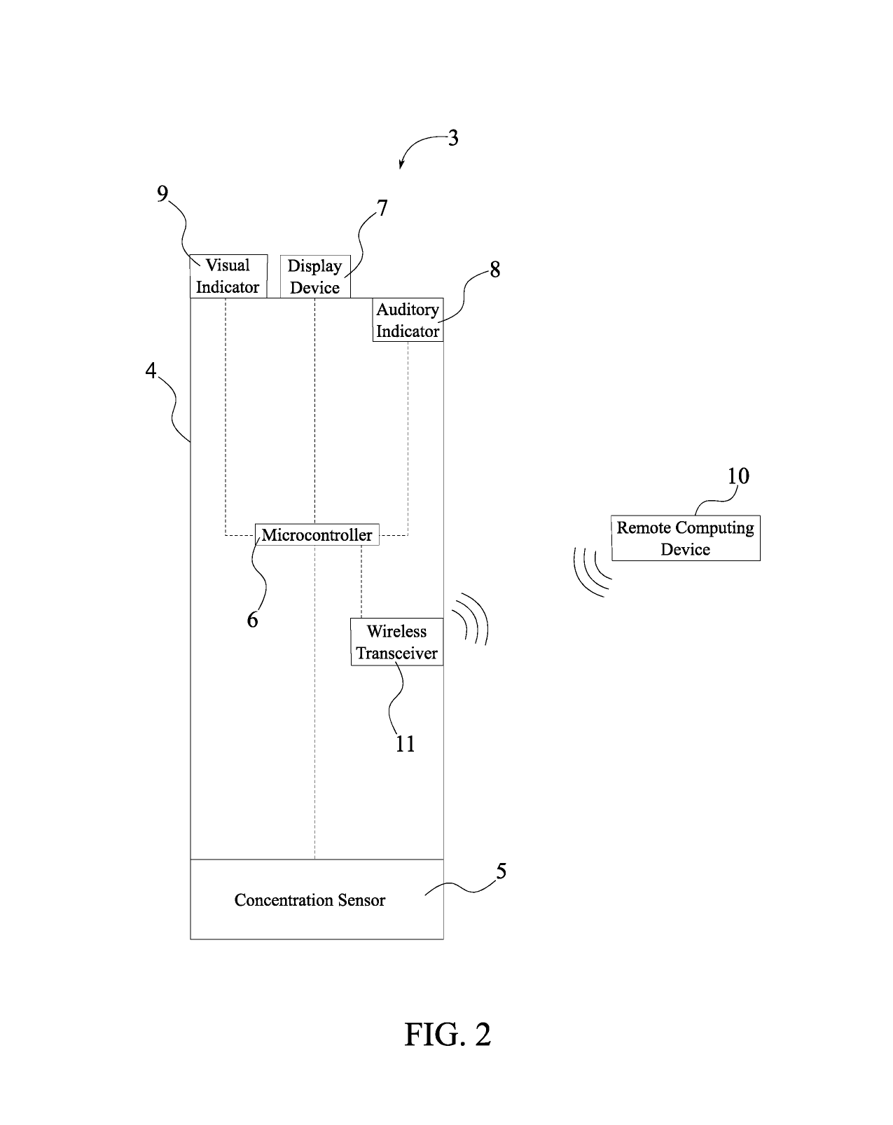

The concentration monitoring device 3 comprises a device housing 4, a concentration sensor 5, a microcontroller 6, and a display device 7, shown in FIG. 2. The device housing 4 supports and protects the electronic components of the present invention from the elements and from degradation due to contact with the sanitation solution 2. The concentration sensor 5 measures the quantity of sanitation compounds within a quantity of solvent as an electrical signal through electrical conductivity, optical absorbance, titration, specific gravity, or any appropriate concentration measuring methods. The concentration sensor 5 is externally mounted to the device housing 4, such that the concentration sensor 5 is able to effectively interface with the sanitation solution 2 when the concentration sensor 5 is submerged in the sanitation solution 2. The concentration sensor 5 is selectively submerged within the sanitation solution 2 such that the user is able to assess the concentration of the sanitation solution 2, as well as allowing the concentration monitoring device 3 to be removed from the sanitation solution 2. Removing the concentration monitoring device 3 prevents the concentration monitoring device 3 from interfering with any cleaning implements being inserted into or removed from the sanitation solution 2. The microcontroller 6 compares the electrical signal to a control value to obtain a measured concentration value relative to a standardized solution. The standardized solution is a solution containing a known quantity of the sanitizer compound to serve as a baseline for the comparison of the electrical output from the concentration sensor 5. The measured concentration value is then able to be viewed by the user through the display device 7. The display device 7 is externally connected to the device housing 4, such that the user is able to view the display device 7 effectively. The display device 7 is oppositely positioned to the concentration sensor 5 along the device housing 4, such that the display device 7 is able to be viewed above the surface of the sanitation solution 2 while the concentration sensor 5 is submerged in the sanitation solution 2. The concentration sensor 5 and the display device 7 are electronically coupled with the microcontroller 6, such that input signals are received from the concentration sensor 5 and processed with the microcontroller 6 to reflect a concentration value proportional to the input signal. The processed input signal is then outputted to the display device 7 such that the user is able to discern the concentration of the sanitation compound of the sanitation solution 2.

In accordance to the preferred embodiment of the present invention, the present invention comprises a remote computing device 10, shown in FIG. 2. The remote computing device 10 receives measurements from the concentration monitoring device 3 to display the measurement values to a user. The remote computing device 10 may include, but is not limited to, personal computers, laptops, smart phones, tablets, or any other appropriate computing device for the user to interface with and view the concentration of the sanitation compound within the sanitation solution 2. The microcontroller 6 is communicatively coupled with the remote computing device 10 to allow a user to view the output concentration value remotely, as well as, store these values on an external storage device. The concentration values are then able to be analyzed on the remote computing device 10 to assess concentration over time or other analytics, such that users would be better able to predict when to refresh the sanitation solution 2 with the corresponding sanitation compound.

In accordance to the preferred embodiment of the present invention, the present invention comprises an auditory indicator 8, detailed in FIG. 1 and FIG. 2. The auditory indicator 8 emits an audible alert to signal nearby hearing-capable persons that deviations in the concentration of the sanitation compound from the pre-determined sanitation concentration range are occurring and need to be addressed. The auditory indicator 8 is internally mounted to the device housing 4, such that the electrical components of the present invention are not exposed to prevent contact with and damage from the sanitation solution 2. The auditory indicator 8 is electronically connected with the microcontroller 6 in order for the microcontroller 6 to initiate the auditory alert when the concentration of the sanitation compound deviates from the pre-determined sanitation concentration range.

Further in accordance to the preferred embodiment of the present invention, the present invention comprises a visual indicator 9, shown in FIG. 1 and FIG. 2. The visual indicator 9 notifies users observing the present invention if there is a deviation in the concentration of the sanitation compound from the pre-determined sanitation concentration range. The visual indicator 9 is externally mounted to the device housing 4 to allow the user to easily observe the concentration indication of the visual indicator 9. The visual indicator 9 is electronically coupled with the microcontroller 6 in order for the microcontroller 6 to illuminate the visual indicator 9 when the concentration of the sanitation compound deviates from the pre-determined sanitation concentration range. The visual indicator 9 is preferred to be a light emitting diode (LED); however, the visual indicator 9 is any indicator that the user is able to assess at a glance.

In some embodiments of the present invention, the present invention comprises a wireless transceiver 11, detailed in FIG. 2. The wireless transceiver 11 allows for the transmission of the concentration of the sanitation solution 2 over a wireless network to the remote computing device 10. The wireless transceiver 11 is internally mounted to the device housing 4, such that the device housing 4 protects the wireless transceiver 11 from the sanitation solution 2. The wireless transceiver 11 is electronically coupled with the microcontroller 6 to transfer data between wireless transceiver 11 and the microcontroller 6. The wireless transceiver may include, but is not limited to, Bluetooth, Wi-Fi, radio frequency, or any other appropriate wireless transmission protocols. The microcontroller 6 is communicatively coupled with the remote computing device 10 through the wireless transceiver 11 to transfer data between the remote computing device 10 and the microcontroller 6. In some more specific embodiments of the present invention, the remote computing device 10 is a plurality of computing devices to allow the concentration of the sanitation compound within the sanitation solution 2 to be viewed by a plurality of users, such as kitchen staff, sanitation staff, managers, system administrators, and establishment owners. In some embodiments of the present invention a central server is utilized to manage the acquisition of the data for the concentration of the sanitation compound to assess concentration over time or other appropriate analytics. The central server then relays the data and data analysis to the remote computing device 10 to be viewed and assessed by a user or a plurality of users.

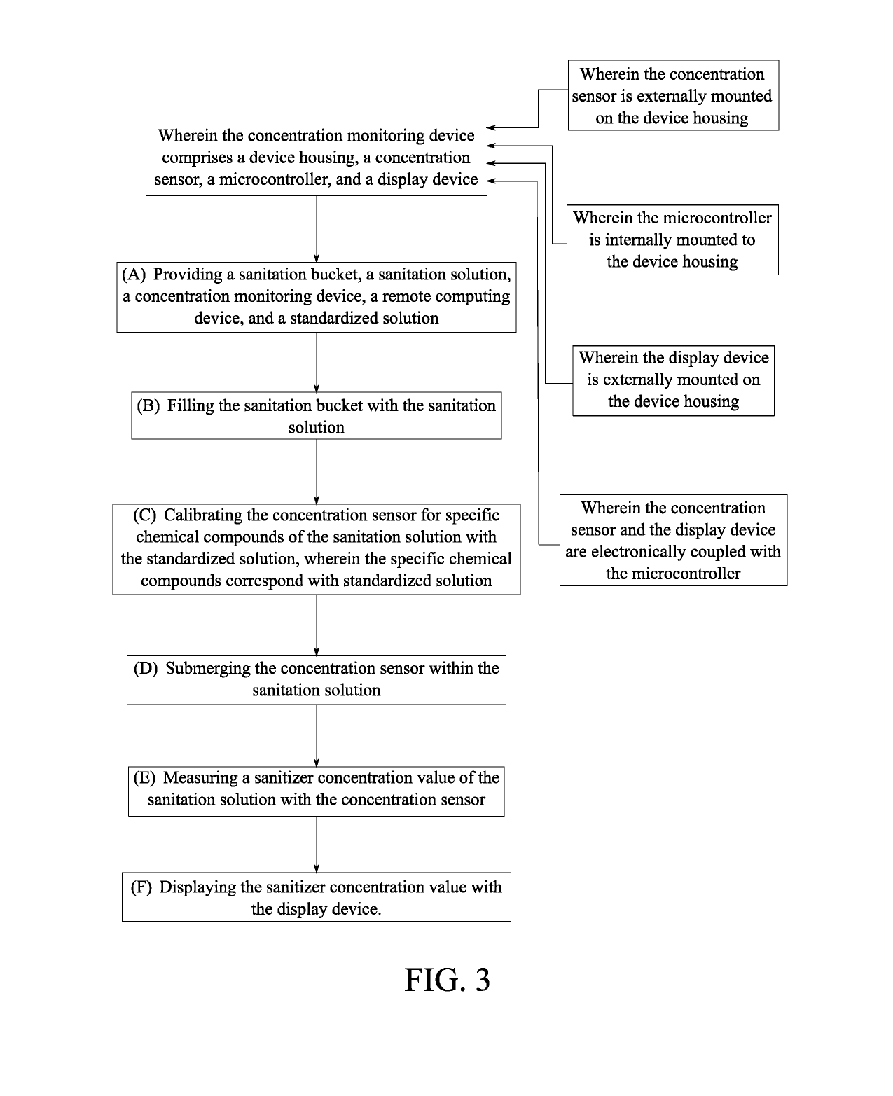

In accordance to the method for monitoring sanitation solutions, the aforementioned system for monitoring sanitation solutions and a standardized solution are necessary (Step A), detailed in FIG. 3. The standardized solution comprises a known concentration of the respective sanitation compound that the sanitation solution 2 is assessed against. Initially, the sanitation bucket 1 is filled with the sanitation solution 2 (Step B), allowing the user access to the sanitation solution 2 to apply the sanitation solution 2 to the desired surface. The concentration sensor 5 is calibrated for specific chemical compounds of the sanitation solution 2 with the standardized solution to obtain an accurate concentration measurement for the sanitation solution 2, wherein the specific chemical compounds correspond with the standardized solution (Step C). Thus, the concentration sensor 5 is able to generate an output signal for the concentration of the sanitation compound in the sanitation solution 2 relative to concentration of the sanitation compound in the standardized solution. The concentration sensor 5 is then submerged within the sanitation solution 2 (Step D) and a sanitizer concentration value of the sanitation solution 2 is measured with the concentration sensor 5 (Step E). The sanitizer concentration value is displayed with the display device 7, such that the user is able to view the sanitation concentration value of the sanitation solution 2 (Step F).

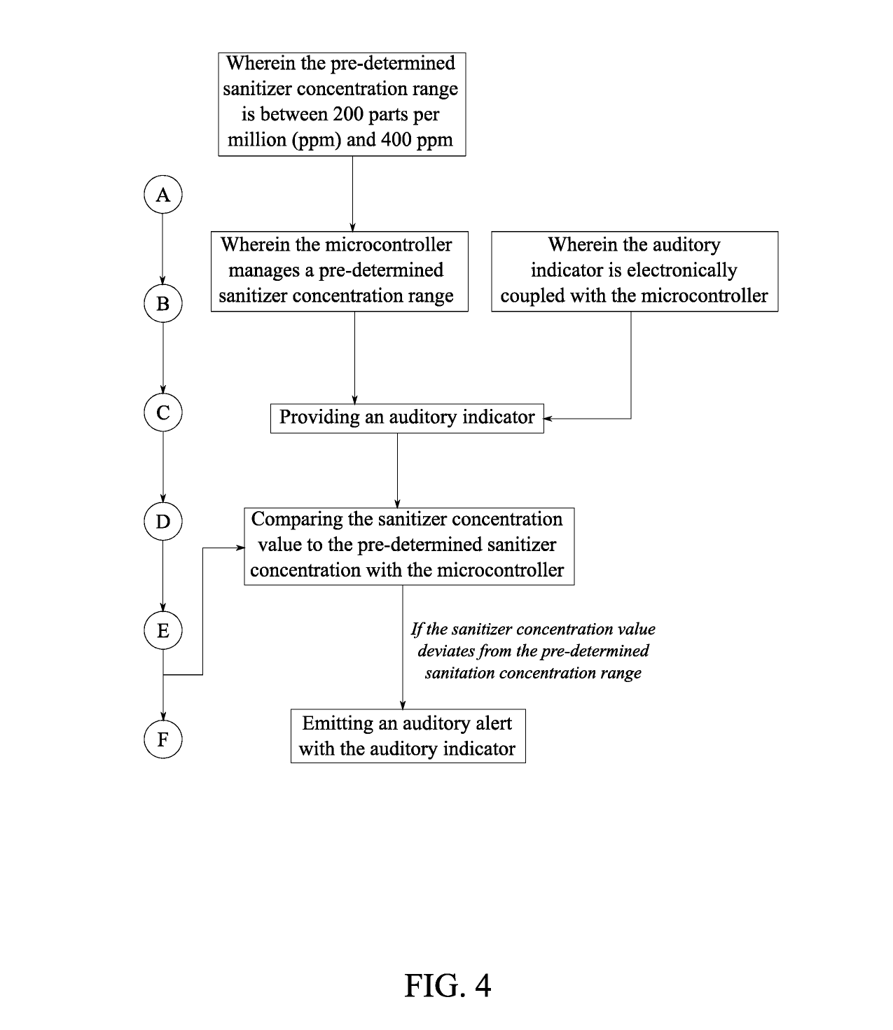

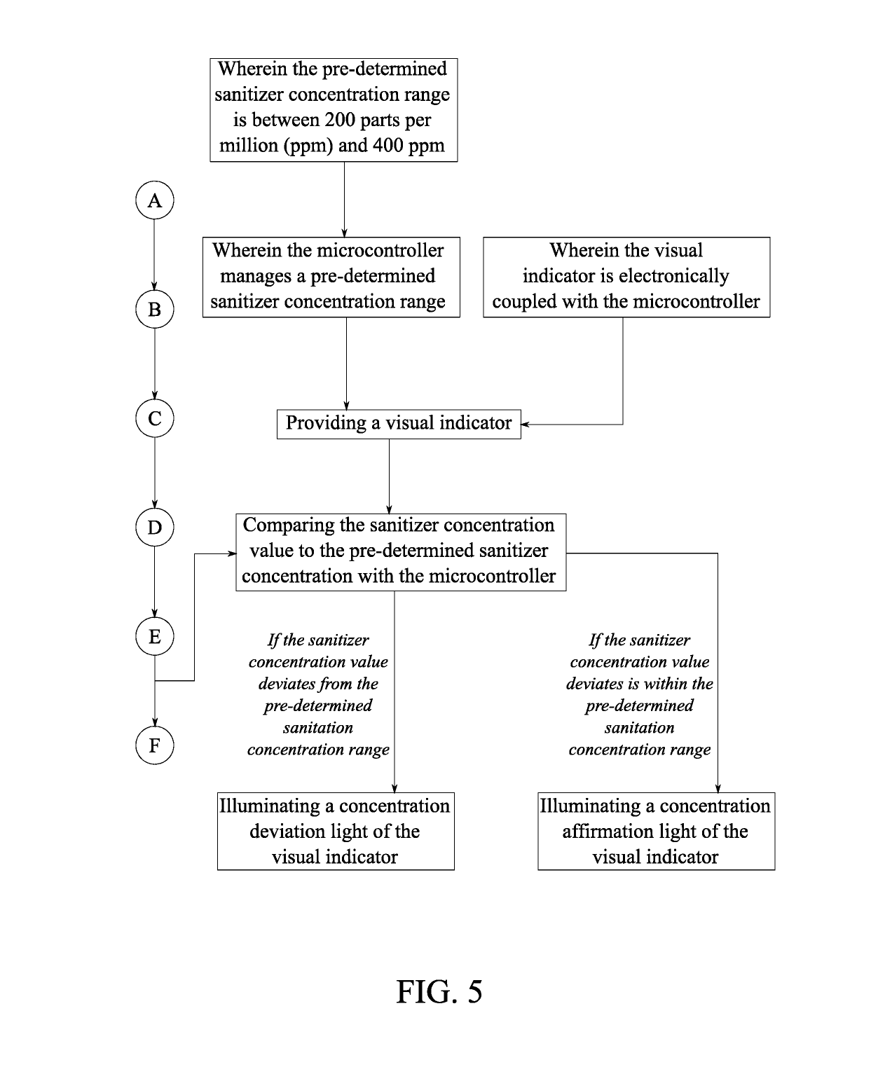

For a more specific embodiment, the microcontroller 6 manages a pre-determined sanitizer concentration range, in accordance to FIG. 4 to FIG. 6. The pre-determined concentration range is a range of concentrations which the sanitation solution 2 is effective or required by law to clean disinfect, and/or sanitize a surface. The pre-determined concentration range is preferred to be between 200 parts per million (ppm) and 400 ppm to ensure the sanitation solution 2 is effective when applied to a desired surface. Subsequent to Step E, the sanitizer concentration value is compared to the pre-determined sanitizer concentration range with the microcontroller 6. In some embodiments, wherein the present invention comprises an auditory indicator 8, if the sanitizer concentration value deviates from the pre-determined sanitation concentration, an auditory alert is emitted to notify any near-by hearing-capable persons, shown in FIG. 4. Similarly in some other embodiments wherein the present invention comprises a visual indicator 9, if the sanitizer concentration value deviates from the pre-determined sanitation concentration, a concentration deviation light of the visual indicator 9 is illuminated, shown in FIG. 5. The concentration deviation light may be color coded as red to signify that the concentration of the sanitation solution 2 needs to be addressed by a user. Additionally, in some more specific embodiments, a concentration affirmation light of the visual indicator 9 is illuminated, if the sanitation concentration value is within the pre-determined sanitation concentration range. The concentration affirmation light may be color coded as green to signify that the concentration of the sanitation solution 2 is within acceptable bounds of the pre-determined sanitation concentration range.

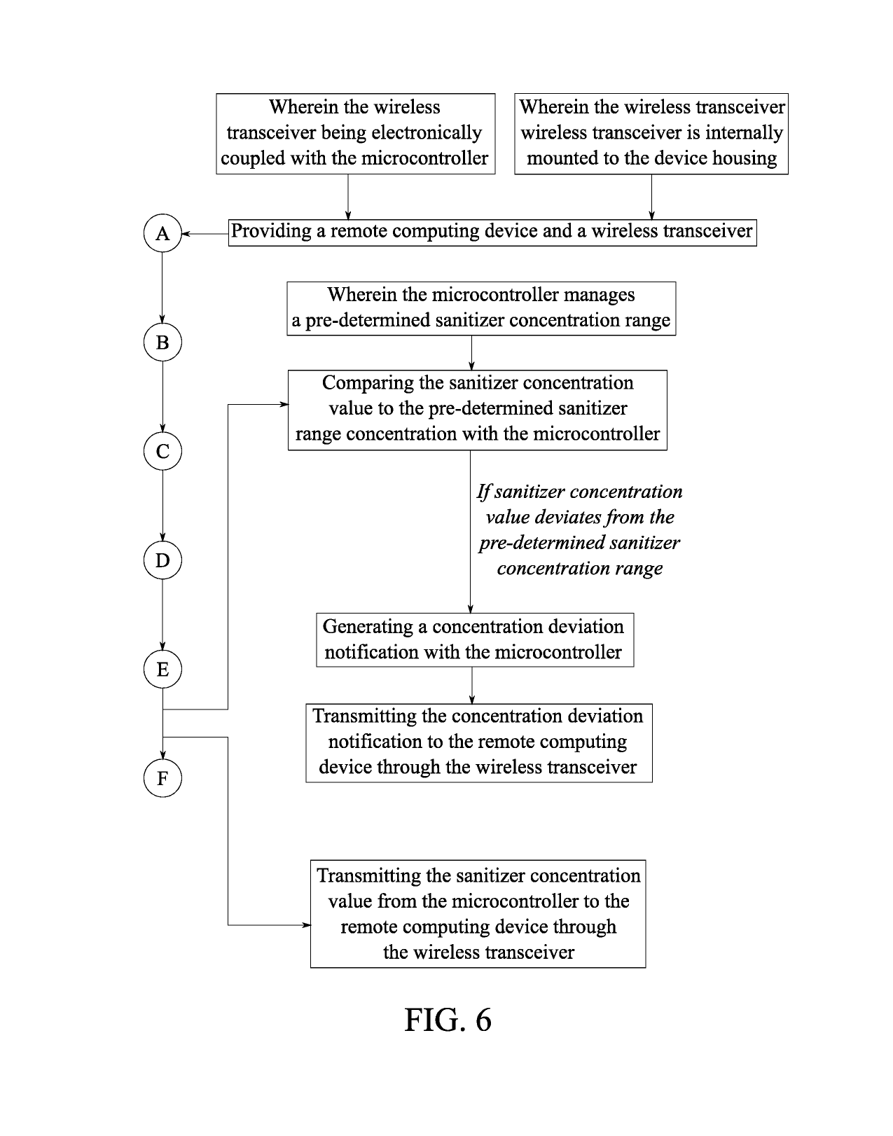

In some embodiments, wherein the present invention comprises the remote computing device 10 and the wireless transceiver 11, the sanitizer concentration value is transmitted from the microcontroller 6 to the remote computing device 10 through the wireless transceiver 11, subsequent to Step E, detailed in FIG. 6. In some more specific embodiments of the present invention, a concentration deviation notification is generated if the sanitizer concentration value deviate from the pre-determined sanitizer concentration range. Subsequently, the concentration deviation notification is transmitted to the remote computing device 10 through the wireless receiver.

Although the invention has been explained in relation to its preferred embodiment, it is to be understood that many other possible modifications and variations can be made without departing from the spirit and scope of the invention as hereinafter claimed.

What is claimed is:

1. A system for monitoring sanitation solutions comprises:
a sanitation bucket;
a sanitation solution;
a concentration monitoring device;
the concentration monitoring device comprises a device housing, a concentration sensor, a microcontroller, and a display device;
the sanitation bucket being filled with the sanitation solution;
the concentration sensor being externally connected to the device housing;
the display device being externally connected to the device housing;
the microcontroller being internally mounted to the device housing;
the display device being oppositely positioned to the concentration sensor along the device housing;
the concentration sensor being selectively submerged within the sanitation solution; and
the concentration sensor and the display device being electronically coupled with the microcontroller.

2. The system for monitoring sanitation solutions, as claimed in claim 1, comprises:
an auditory indicator;
the auditory indicator being internally mounted to the device housing; and
the auditory indicator being electronically coupled with the microcontroller.

3. The system for monitoring sanitation solutions, as claimed in claim 1, comprises:
a visual indicator;
the visual indicator being externally mounted to the device housing; and
the visual indicator being electronically coupled with the microcontroller.

4. The system for monitoring sanitation solutions, as claimed in claim 1, wherein the visual indicator is a light emitting diode (LED).

5. The system for monitoring sanitation solutions, as claimed in claim 1, comprises:
a remote computing device;
a wireless transceiver;
the wireless transceiver being internally mounted to the device housing;
the wireless transceiver being electronically coupled with the microcontroller; and
the microcontroller being communicatively coupled with the remote computing device through the wireless transceiver.

6. A method for monitoring sanitation solutions comprises the steps of:
(A) providing a sanitation bucket, a sanitation solution, a concentration monitoring device, a remote computing device, and a standardized solution, wherein the concentration monitoring device comprises a device housing, a concentration sensor, a microcontroller, and a display device, wherein the concentration sensor is externally mounted on the device housing, wherein the microcontroller is internally mounted to the device housing, wherein the display device is externally mounted on the device housing, and wherein the concentration sensor and the display device are electronically coupled with the microcontroller;
(B) filling the sanitation bucket with the sanitation solution;
(C) calibrating the concentration sensor for specific chemical compounds of the sanitation solution with the standardized solution, wherein the specific chemical compounds correspond with standardized solution;
(D) submerging the concentration sensor within the sanitation solution;
(E) measuring a sanitizer concentration value of the sanitation solution with the concentration sensor; and
(F) displaying the sanitizer concentration value with the display device.

7. The method for monitoring sanitation solutions, as claimed in claim 6, comprises the steps of:
providing an auditory indicator, wherein the auditory indicator is electronically coupled with the microcontroller, wherein the microcontroller manages a pre-determined sanitizer concentration range;
comparing the sanitizer concentration value to the pre-determined sanitizer concentration with the microcontroller, subsequent to Step E; and
emitting an auditory alert with the auditory indicator, if the sanitizer concentration value deviates from the pre-determined sanitation concentration.

8. The method for monitoring sanitation solutions, as claimed in claim 7, wherein the pre-determined sanitizer concentration range is between 200 parts per million (ppm) and 400 ppm.

9. The method for monitoring sanitation solutions, as claimed in claim 6, comprises the steps of:
providing a visual indicator, wherein the visual indicator is electronically coupled with the microcontroller, wherein the microcontroller manages a pre-determined sanitizer concentration range;
comparing the sanitizer concentration value to the pre-determined sanitizer concentration range with the microcontroller; and
illuminating a concentration deviation light of the visual indicator,
if the sanitizer concentration value deviates from the pre-determined sanitation concentration range.

10. The method for monitoring sanitation solutions, as claimed in claim 9, comprises the steps of:
illuminating a concentration affirmation light of the visual indicator,
if the sanitizer concentration value is within the pre-determined sanitation concentration range.

11. The method for monitoring sanitation solutions, as claimed in claim 6, comprises the steps of:
providing a remote computing device and a wireless transceiver, wherein the wireless transceiver wireless transceiver is internally mounted to the device housing and wherein the wireless transceiver being electronically coupled with the microcontroller; and
transmitting the sanitizer concentration value from the microcontroller to the remote computing device through the wireless transceiver.

12. The method for monitoring sanitation solutions, as claimed in claim 11, comprises the steps of:
wherein the microcontroller manages a pre-determined sanitizer concentration range;
comparing the sanitizer concentration value to the pre-determined sanitizer range concentration with the microcontroller;
generating a concentration deviation notification with the microcontroller,
if sanitizer concentration value deviates from the pre-determined sanitizer concentration range; and
transmitting the concentration deviation notification to the remote computing device through the wireless transceiver.
